(19) United States
(12) Patent Application Publication
(10) Pub. No.: US 2024/0043555 A1
(43) Pub. Date: Feb. 8, 2024

(54) METHOD FOR TREATING ACUTE AND CHRONIC KIDNEY INJURY

(71) Applicant: ICHILOV TECH LTD., Tel-Aviv (IL)

(72) Inventors: Idit F. SCHWARTZ, Tel-Aviv (IL); Nadir ARBER, Tel-Aviv (IL); Doron SCHWARTZ, Tel-Aviv (IL); Lior YANKELSON, Tel-Aviv (IL)

(21) Appl. No.: 18/229,821

(22) Filed: Aug. 3, 2023

(30) Foreign Application Priority Data
Aug. 4, 2022 (GB) 2211356.7

(51) Int. Cl. C07K 16/28; A61K 31/7105; A61P 13/12

(52) U.S. Cl. CPC C07K 16/2896; A61K 31/7105; A61P 13/12; C07K 2317/76

(57) ABSTRACT

Provided herein is a method for treating or preventing a kidney disease or injury in a subject in need thereof, the method including administering to the subject a therapeutically effective amount of pharmaceutical composition including a CD24 inhibitor. Further provided is a method for inhibiting a damage associated molecular pattern (DAMP) in a subject, including administering to the subject a therapeutically effective amount of pharmaceutical composition including a CD24 inhibitor.

### CROSS-REFERENCE TO RELATED APPLICATIONS

This application claims the benefit of priority of GB Patent Application No. 2211356.7, filed Aug. 4, 2022, the contents of which are all incorporated herein by reference in their entirety.

### FIELD OF THE INVENTION

The present invention relates to methods for treating renal diseases or injuries.

### BACKGROUND

Kidney disease (KD), also known as renal disease, is a progressive loss in renal function over a period of months or years. In particular, kidney disease (KD) is a major U.S. public health concern with recent estimates suggesting that more than 26 million adults in the U.S. have the disease including chronic kidney disease (CKD). The primary causes of KD include diabetes and high blood pressure, which are responsible for up to two-thirds of the cases. In recent years, the prevalence of KD has increased due to a rising incidence of diabetes mellitus, hypertension (high blood pressure) and obesity, and also due to an aging population.

Kidney disease include both chronic kidney disease as well as acute injury-induced kidney disease (AKI).

In the case of CKD, patients have an increased risk of death from cardiovascular events because CKD is thought to accelerate the development of heart disease. CKD patients generally have cardiac-specific mortality rates many times higher than age- and sex-matched non-CKD populations, and it has been suggested that the pathological heart-kidney interactions are bidirectional in nature.

During acute kidney injury (AKI) an acute decrease in renal function occurs. Inflammation, parenchymal cell loss, and nephron loss are features of AKI that may eventually lead to tubulointerstitial fibrosis. Mortality is as high as 50% and has changed little over the past three decades. Even mild decrements in renal function which do not necessitate dialytic therapy are recognized as being associated with poor patient prognosis. Patients with AKI have a higher risk for developing CKD and reaching end stage renal disease (ESRD) requiring renal replacement therapy and harbor an increased long-term mortality risk. This AKI to CKD progression has been recognized as one of the most pressing unmet needs in renal medicine. Currently, no satisfactory treatment has been demonstrated to be effective for preventing AKI or reducing the high mortality and progression to CKD. The pathogenesis of AKI is characterized by renal tubular cell death which is followed by tubular dedifferentiation, proliferation, and regeneration. During AKI, a noninfectious inflammatory response is induced by release of chemokines from sub-lethally injured cells and damage-associated molecular patterns (DAMPs) from dying cells. DAMPs represent intracellular components released from necrotic cells. DAMPs bind to and activate membrane-bound Toll-like receptors, instigating a host-specific cascade during the early defense phase of innate immunity. This cascade perpetuates the inflammatory response and aggravates tissue injury.

CD24 is a small (80 amino acids) glycosyl phosphoinositol-anchored protein that is expressed on both hematopoietic cells and non-hematopoietic cells, including neural cells, epithelial cells, keratinocytes, muscle cells and in many types of cancer cells. As a rule, CD24 tends to be expressed at higher levels in progenitor cells and metabolically active cells and to a lesser extent in terminally differentiated cells. Diverse immunological functions for CD24 have been reported: on activated B cells, CD24 functions as a T-cell co-stimulator for CD4+ T cell clonal expansion. Likewise, CD24 is highly expressed on immature T cells and is weakly expressed on peripheral T cells but it is upregulated in activated T cells. A functional CD24 gene is required for optimal homeostatic proliferation of T cells in a lymphopenic host. Interestingly, CD24 was demonstrated to be associated with a variety of DAMPs, such as high-mobility group box protein-1, Heat-shock proteins and nucleolins. The role of CD24 in modulation of tissue injury is confusing. In a mouse model of acetaminophen-induced liver necrosis CD24 negatively regulates the immune response to proteins released by damaged cells, resulting in attenuation of tissue injury. In contrast, CD24 has been shown to aggravate acute liver injury in autoimmune hepatitis by promoting interferon gamma generation by CD4+ T cells. In the kidney, CD24 is expressed on immature cells and disappears after they have reached their final stage of differentiation. CD24 is absent in normal mature renal tissue. The involvement of CD24 in the pathogenesis of AKI is yet to be resolved.

### SUMMARY

The present invention, in some embodiments, is based, in part, on the surprising findings that CD24 inhibition attenuated decrease in renal function and histologic injury, and lowered serum and/or renal levels of proinflammatory cytokines, e.g., Interleukin-10, interferon gamma, and tumor necrosis factor alpha. Further, renal, and systemic IL-33 upregulation was augmented, and CD24 omission, e.g., in a knockout model organism, resulted in increased splenic margination and renal infiltration of regulatory T cells (Tregs). Specifically, the administration of anti-CD24 antibodies to an acute kidney injury (AKI) murine model, resulted in an attenuated decrease in renal function and histologic injury compared to non-treated AKI wild type controls. The inhibition of CD24 activity, for example by using neutralizing antibodies as disclosed herein, is an attractive modality for AKI therapy.

The present invention, in some embodiments, is based, in part, on the findings that absence of CD24 (e.g., by knockout) has attenuated the severity of an induced acute or chronic kidney disease in mice. Therefore, the inhibition of CD24 activity, for example by using neutralizing antibodies as disclosed herein, is generally attractive to kidney disease or injury, including both AKI and chronic kidney disease.

According to a first aspect, there is provided a method for treating or preventing a kidney disease or injury in a subject in need thereof, the method comprising administering to the subject a therapeutically effective amount of pharmaceutical composition comprising a CD24 inhibitor, thereby, treating or preventing a kidney disease or injury in the subject.

According to another aspect, there is provided a method for inhibiting a damage associated molecular pattern (DAMP) in a subject in need thereof, the method comprising administering to the subject a therapeutically effective amount of a pharmaceutical composition comprising a CD24 inhibitor and a pharmaceutically acceptable carrier, thereby inhibiting a DAMP in the subject.

In some embodiments, the kidney disease or injury comprises any one of: acute kidney injury (AKI), and chronic kidney disease.

In some embodiments, the treating or preventing comprises reducing a level of at least one cytokine selected from the group consisting of: interleukin-10 (IL-10), interferon gamma (INF-γ), tumor necrosis factor alpha (TNFα), and any combination thereof, in the subject.

In some embodiments, the treating or preventing comprises increasing a level of IL-33, in the subject.

In some embodiments, the treating or preventing comprises reducing levels of serum creatinine, blood urea nitrogen (BUN), urinary albumin to creatinine ratio, or any combination thereof, in the subject.

In some embodiments, the treating or preventing comprises increasing splenic margination of T regulatory cells (Tregs), in the subject.

In some embodiments, the treating or preventing comprises increasing renal infiltration of Tregs, in the subject.

In some embodiments, the Tregs are Foxp3+ Tregs.

In some embodiments, the AKI comprises acute tubular necrosis (ATN).

In some embodiments, the pharmaceutical composition further comprises a pharmaceutically acceptable carrier.

In some embodiments, the CD24 inhibitor is a polypeptide or a polynucleotide.

In some embodiments, the polypeptide is an antibody.

In some embodiments, the antibody is a blocking or inhibitory antibody.

In some embodiments, the antibody is not cytotoxic.

In some embodiments, the polynucleotide is an RNA interfering polynucleotide.

In some embodiments, the treating or preventing comprises reducing release of pro-immunogenic cellular components from damaged cells, in the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by reference to the figures and by study of the following detailed description.

### BRIEF DESCRIPTION OF THE FIGURES

FIGS. 1A-1F are micrographs, and graphs showing the involvement of CD24 in renal function in an acute kidney injury model. (1A) is micrographs showing renal CD24 expression detected by immunohistochemistry in WT and CD24^{-/-} mice with FA-AKI. Progressive positive staining for CD24 restricted to the distal tubular epithelial cells was observed in the WT FA-AKI mice while it was absent in the corresponding CD 24^{-/-} animals. Four animals per group. Original magnification 40×. Scale bars, 50 (1B-1D) are graphs showing kidney function in response to folic acid injury. Renal function was assessed by plasma creatinine (1B), blood urea nitrogen (BUN; 1C), and neutrophil gelatinase-associated lipocalin (NGAL; 1D) levels. Renal failure was significantly attenuated in the folic acid acute kidney injury CD24^{-/-} (FA-AKI^{-/-}) mice compared with the corresponding WT animals. Data present the mean±standard error of the mean (SEM) of eight mice per group. *P<0.05 versus the corresponding knockout (K/O) experimental group. (1E) is micrographs showing decreased histologic injury in kidneys from K/O mice exposed to FA. Representative images of Masson staining and quantification. Original magnification 40×. Scale bars, 50 μm. (1F) is a vertical bar graph showing the mean±SEM of renal injury score measured for six mice per each of the experimental groups. *P<0.05 versus the corresponding WT animal.

FIGS. 2A-2B are micrographs of western blot analysis and a vertical bar graph showing that CD 24^{-/-} mice exhibit increased early apoptotic activity which is attenuated during the late phases of FA-AKI compared to the corresponding WT animals. (2A-2B) are representative western blot and densitometric analysis showing regulation of: (2A) caspase 3 (pro-apoptotic) and (2B) Bcl-XL (anti-apoptotic) proteins in K/O and WT mice following folic acid administration (FA-AKI). Data represent the mean±SEM of 3 different experiments *P<0.05 vs. the corresponding WT animals.

FIGS. 3A-3B are vertical bar graphs showing the dichotomic behavior of INF-γ and IL-10 during FA-AKI in CD 24^{-/-} mice compared to WT animals. The increment in plasma INF-γ and IL-10 was attenuated in the K/O mice. Plasma INF-γ, and IL-10, were quantified by ELISA. Results represent mean±SEM of seven animals per group. *P<0.05 vs. the corresponding WT mice.

FIGS. 4A-4B are micrographs of western blot analysis and vertical bar graphs showing the dichotomic behavior of renal tubular protein expression of TNFα and IL-33 during FA-AKI in CD 24^{-/-} mice compared to WT animals. The increment in renal tubular TNFα was attenuated while IL-33 was increased in CD 24^{-/-} mice compared to WT during FA-AKI. (4A-4B) are representative western blot and densitometric analysis showing regulation of: (4A) TNFα and (4B) IL-33 protein level following the administration of folic acid in CD 24^{-/-} mice. Data represent mean±SEM of 3 different experiments. *P<0.05 vs. the corresponding WT animals.

FIGS. 5A-5B are micrographs and a vertical bar graph showing that Tregs are more abundant in CD24^{-/-} mice after folic acid (FA-AKI). Presence of Foxp3⁺ T cells, were detected by immunohistochemistry (5A), in the germinal center (GC) and marginal zone (MZ) of spleens, and kidneys harvested from CD24^{-/-} and WT mice 7 days after the administration of folic acid. Four animals per group. Original magnification 40×. Scale bars, 50 μm. Foxp3+ T cells were counted per field of view in WT and K/O mice (5B). *P<0.05 vs. the corresponding WT animals.

FIGS. 6A-6B are micrographs and a vertical bar graph showing decreased histologic injury in kidneys from WT mice which were exposed to FA and treated with anti-CD 24 antibodies. (6A) shows a representative image of Masson staining and quantification of day 7 following folic acid. Original magnification 40×. Scale bars, 500 μm. (6B) is a vertical bar graph showing the mean±SEM of renal injury score measured for six mice per group. *P<0.05 vs. the corresponding WT animal.

FIGS. 7A-7B are graphs showing kidney function in response to administration of anti-CD 24 antibodies to WT mice subjected to folic acid injury. Renal function was assessed by plasma creatinine (7A) and BUN (7B) levels. Renal failure was significantly attenuated in the antibodies treated FA-AKI mice compared with the corresponding untreated WT animals. Data represent mean±SEM of eight mice per group. *P<0.05 vs the corresponding untreated experimental group.

FIG. 8 is micrographs of western blot analysis and a vertical bar graph showing the incremental increase in renal tubular IL-33 in WT mice treated with anti-CD 24 antibodies compared to untreated WT animals during FA-AKI. Representative western blot and densitometric analysis showing regulation of IL-33 protein level following the administration of folic acid (FA). Data represent mean±SEM of 3 different experiments. *P<0.05 vs. the corresponding WT animals.

FIGS. 9A-9C are micrographs of immunohistochemistry analysis showing renal CD24 expression detected by in patients with acute tubular necrosis (ATN). Progressive positive staining for CD24 restricted to the distal tubular epithelial cells was observed in: (9A) a patient with ATN due to long standing hypovolemia and (9B) a patient with delayed graft function following deceased donor kidney transplantation. (9C) Negative staining in normal renal tissue which was removed along with renal cell carcinoma.

FIG. 10 includes a graph showing serum creatinine levels as measured in wild type (WT) and CD24 knockout (K/O). Measurements were conducted at time 0 (CTL) or after 1 month of adenine administration, a common and accepted model of chronic renal failure (CRF).

FIG. 11 includes a graph showing the ratio of urinary albumin to creatinine as measured in the WT and K/O mice of FIG. 10.

### DETAILED DESCRIPTION

According to a first aspect, there is provided a method for treating or preventing a kidney disease or injury in a subject in need thereof.

According to another aspect, there is provided a method for treating or preventing acute kidney injury (AKI) in a subject in need thereof.

According to another aspect, there is provided a method for treating or preventing chronic kidney disease (CKD) in a subject in need thereof.

According to another aspect, there is provided a method for inhibiting a DAMP associated disease or condition in a subject in need thereof.

In some embodiments, the method comprises administering to the subject a pharmaceutical composition comprising a therapeutically effective amount of a CD24 inhibitor.

In some embodiments, the method comprises reducing or attenuating renal dysfunction in the subject. Determining renal function can be performed by histological analysis, as would be apparent to one of ordinary skill in the art, and as exemplified herein below. In some embodiments, the method comprises reducing the serum level, the renal level, or both, of a cytokine selected from: IL-10, INF-γ, TNFα, or any combination thereof, in the subject. In some embodiments, the method comprises reducing the serum level of creatinine, BUN, or both, in the subject. In some embodiments, the method comprises reducing the urinary albumin to creatinine ratio of the subject.

In some embodiments, the method further comprises a step of determining the serum level, the renal level, or both, of a cytokine selected from: IL-10, INF-γ, TNFα, or any combination thereof, in the subject. In some embodiments, determining the serum level of IL-10, INF-γ, or both, is performed at least 1 day, at least 2 days, or at least 3 days, after AKI is initiated, or any value and range therebetween. Each possibility represents a separate embodiment of the invention. In some embodiments, determining the serum level of IL-10, INF-γ, or both, is performed, at least 1 day before, at least 2 days before, or at least 3 days before loss of kidney function develops.

In some embodiments, the method further comprises a step of determining the serum level of creatinine, BUN, urinary albumin to creatinine ratio, or any combination thereof, in the subject. In some embodiments, determining the serum level of creatinine, BUN, or both, is performed at least 5, at least 6, or at least 7 days after AKI is initiated, or any value and range therebetween. Each possibility represents a separate embodiment of the invention. In some embodiments, determining the serum level of creatinine, BUN, or both, is performed on the day, at least 1 day after, at least 2 days after, or at least 3 days after loss of kidney function develops.

In some embodiments, the method comprises inhibiting or preventing a renal caspase 3 overexpression or peak expression. In some embodiments, inhibiting or preventing the renal caspase 3 overexpression or peak expression is for a period ranging from day 1 to day 4, day 5, or day 7, after AKI is initiated.

In some embodiments, the method further comprises a step of determining renal level of caspase 3. In some embodiments, determining the renal level of caspase 3, is performed at 1 day at most, 2 days at most, or 3 days at most, after AKI is initiated, or any value and range therebetween. Each possibility represents a separate embodiment of the invention. In some embodiments, determining the renal level of caspase 3, is performed at least 6 days before, at least 5 days before, at least 4 days before, or at least 3 days before loss of kidney function develops.

In some embodiments, loss of kidney function develops about 7 days after AKI is initiated. In some embodiments, loss of kidney function development refers to the time point wherein loss of function is evident and would be determined by a physician. In some embodiments, during the first 7 days after AKI is initiated a subject is yet to be diagnosed or determined as being afflicted with loss of kidney function. In some embodiments, loss of kidney function is detectable only after at least 6 days, at least 7 days, or at least 8 days after AKI is initiated, or any value and range therebetween. Each possibility represents a separate embodiment of the invention.

In some embodiments, the determining step is performed in the subject or in a sample derived or obtained from the subject. In some embodiments, the sample comprises any bodily fluid, cell, tissue, biopsy, organ, or a combination thereof, derived or obtained from the subject. In some embodiments, the determining step is performed in vivo or in vitro. In some embodiments, in vitro comprises or is in a test tube or in a plate.

In some embodiments, any one of the terms “reduced”, “reducing”, “increased” and “increasing” are compared to a control. In some embodiments, the reduction is at least a 10, 15, 20, 25, 30, 35, 40, 45, 50, 55, 60, 65, 70, 75, 80, 85, 90, 95, 97, 99 or 100% reduction. Each possibility represents a separate embodiment of the invention. In some embodiments, the increasing is at least a: 10, 20, 25, 30, 40, 50, 60, 70, 75, 80, 90, 100, 150, 200, 250, 300, 350, 400, 450, 500, 600, 700, 800, 900 or 1000% increase. Each possibility represents a separate embodiment of the invention.

In some embodiments, the method comprises increasing splenic leukocytes margination in the subject. In some embodiments, the method comprises increasing renal infiltration of immunoregulatory cells in the subject. In some embodiments, the method comprises increasing the serum level, the renal level, or both, of IL-33, in the subject. In some embodiments, the immunoregulatory cells are T regulatory cells. In some embodiments, the T regulatory cells are positive for the CD3 marker (CD3+) and for the Foxp3 marker (Foxp3+).

In some embodiments, the method further comprises a step of determining the serum level, the renal level, or both, of IL-33, in the subject. In some embodiments, the method further comprises a step of determining splenic margination in the subject. In some embodiments, the method further comprises a step of determining renal infiltration of immunoregulatory cells in the subject.

In some embodiments, determining splenic margination is performed at least 6 days, at least 7 days, or at least 8 days after AKI is initiated, or any value and range therebetween. Each possibility represents a separate embodiment of the invention. Methods for specific cell classification are common and would be apparent to a skilled artisan, and may include, but is not limited to classification based on membranal markers, such as by flow cytometry or fluorescent activated cell sorting (FACS), as exemplified hereinbelow.

In some embodiments, reducing or reduced is by at least 5%, at least 10%, at least 15%, at least 25%, at least 35%, at least 50%, at least 65%, at least 75%, at least 85%, at least 90%, at least 95%, at least 99%, or 100% compared to control, or any value and range therebetween. Each possibility represents a separate embodiment of the invention. In some embodiments, reducing or reduced is by 1 to 20%, 5 to 45%, 10 to 75%, 15 to 85%, 1 to 90%, 20 to 100%, or 25 to 95% compared to control. Each possibility represents a separate embodiment of the invention.

In some embodiments, increasing or increased is by at least 5%, at least 15%, at least 25%, at least 50%, at least 75%, at least 100%, at least 200%, at least 350%, at least 500%, at least 650%, at least 750%, at least 1,000%, or any value and range therebetween. Each possibility represents a separate embodiment of the invention. In some embodiments, increased is by 5 to 50%, 25 to 150%, 100 to 450%, 75 to 500%, 250 to 700%, 550 to 1,000%, or 350 to 1,200%. Each possibility represents a separate embodiment of the invention.

In some embodiments, a control comprises or is a healthy subject or a sample derived therefrom. In some embodiments, a control comprises or is an AKI or CKD subject not treated with the herein disclosed CD24 inhibitor or a sample derived therefrom. In some embodiments, a control is a subject afflicted with a non CD24 related kidney disease or injury, or a sample derived therefrom. In some embodiments, a control is a subject afflicted with a non-DAMPs related disease or disorder, or a sample derived therefrom.

In some embodiments, administrating is abdominally administrating. In some embodiments, administrating is subcutaneously administrating. In some embodiments, administrating is intra-peritoneally administrating. In some embodiments, administrating is intravenously administrating. In some embodiments, administrating is systemically administrating.

As used herein, the terms “CD24” or “cluster of differentiation 24” refers to a sialoglycoprotein that is expressed at the surface of most B lymphocytes, and neutrophils, and is encoded by the gene CD24 (Accession number: NP_001278666).

In some embodiments, the CD24 inhibitor is in a composition. In some embodiments, the composition further comprises an acceptable carrier. In some embodiments, the carrier is a pharmaceutically acceptable carrier. In some embodiments, the composition is a pharmaceutical composition. In some embodiments, the composition further comprises a pharmaceutically acceptable carrier, excipient, or adjuvant.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the effect of the composition comprises or is characterized by a reduced level of at least one cytokine. In some embodiments, treatment comprises reducing a level of at least one cytokine. In some embodiments, reducing a level of a cytokine is reducing secretion of the cytokine. In some embodiments, the cytokine is a proinflammatory cytokine. In some embodiments, the cytokine is an anti-inflammatory cytokine. In some embodiments, the cytokine is selected from: interleukin-10 (IL-10), interferon gamma (INF-γ), tumor necrosis factor alpha (TNFα), or any combination thereof.

In some embodiments, composition comprises or is characterized by induction of an increased level of IL-33. In some embodiments, the composition increases a level of IL-33. In some embodiments, the increase is an increase in IL-33 secretion. In some embodiments, the treatment comprises an increase in IL-33 expression.

In some embodiments, the level of a cytokine as disclosed herein refers to the expression level of the cytokine. In some embodiments, the level of a cytokine refers to the expression level in blood of the subject. In some embodiments, the level of a cytokine refers to the expression level in a tissue. In some embodiments, the tissue is renal tissue.

The term “expression” as used herein refers to the biosynthesis of a gene product, including the transcription and/or translation of the gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Methods for determining the expression level of a cytokine as disclosed herein, e.g., ILs, INF-γ, TNFα, are common and would be apparent to one of ordinary skill in the art. Non-limiting examples of methods for determining expression levels include, but are not limited to, RT-PCR, real time-RT-PCR, protein dot blot, RNA in situ hybridization, densitometry, western blot, enzyme-linked immunosorbent assay (ELISA), some of which are exemplified herein below, and/or others.

In some embodiments, the composition comprises or is characterized by a reduced level of serum creatinine, blood urea nitrogen (BUN), urinary albumin to creatinine ratio, or any combination thereof. In some embodiments, the composition induces a reduction in a level of creatinine, BUN, urinary albumin to creatinine ratio, or any combination thereof. In some embodiments, treatment comprises a reduction in a level of creatinine, BUN, urinary albumin, or any combination thereof. In some embodiments, the reduction is a reduction in a level of creatinine. In some embodiments, the reduction is a reduction in BUN. In some embodiments, the reduction is a reduction of urinary albumin to creatinine ratio. Methods for determining the levels of creatinine, BUN, urinary albumin to creatinine, would be apparent to a skilled artisan, e.g., blood test for urea nitrogen levels by standard picrate method, urine sample, or other, such as exemplified herein below.

In some embodiments, the composition comprises or is characterized by an increase of margination. In some embodiments, the composition induces increased margination. In some embodiments, treatment comprises increased margination. As used herein, the term “margination” refers to movement of Tregs through specific organs, which in turn results in discrete intravascular (marginated) pools. In some embodiments, margination is through an organ selected from: spleen, liver, bone marrow, lung, or any combination thereof. In some embodiments, margination comprises or consists of splenic margination.

In some embodiments, the composition comprises or is characterized by an increase of renal infiltration of T regulatory cells (Tregs). In some embodiments, the composition induced an increase of renal infiltration of Tregs. In some embodiments, treatment or prevention comprises an increase of renal infiltration of T regulatory cells (Tregs). In some embodiments, the Tregs are Foxp3+ Tregs.

Movement and/or presence of particular cells in a tissue e.g., margination (for example splenic margination), infiltration (for example of T cells), can be determined according to any methods known in the art. Non-limiting examples for such methods of determination include, but are not limited to, histology, immunohistochemistry, cell-specific stains, cell-component specific stains, such as exemplified hereinbelow, and/or others.

In some embodiments, the CD24 inhibitor is a polypeptide or a polynucleotide. As used herein, the terms “peptide”, “polypeptide”, and “protein” are used interchangeably to refer to a polymer of amino acid residues. In another embodiment, the terms “peptide”, “polypeptide”, and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide”, and “protein” apply to naturally occurring amino acid polymers. In another embodiment, the terms “peptide”, “polypeptide”, and “protein” apply to amino acid polymers in which one or more amino acid residue is an artificial chemical analogue of a corresponding naturally occurring amino acid.

In some embodiments, the polypeptide is an antibody. As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelid, CDR-grafted, multi-specific, bi-specific, catalytic, humanized, fully human, anti-idiotypic and antibodies that can be labeled in soluble or bound form as well as fragments, including epitope-binding fragments, variants, or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulfide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the CD24 inhibitor is an anti-CD24 antibody. In some embodiments, the CD24 inhibitor is a CD24 antagonist. In some embodiments, the antibody is a blocking antibody. In some embodiments, the antibody is a neutralizing antibody. In some embodiments, the antibody binds to a ligand-binding pocket on CD24. In some embodiments, the antibody induces internalization of CD24. In some embodiments, the antibody induces degradation of CD24. In some embodiments, the antibody does not induce cytotoxicity of a CD24 expressing cell. In some embodiments, binding of the antibody to a cell does not induce antibody-mediated cytotoxicity in the bound cell. In some embodiments, the antibody comprises an IgG2 or IgG4 backbone. In some embodiments, the antibody comprises an IgG1 or IgG3 backbone. In some embodiments, the antibody does not comprise an IgG1 or IgG3 backbone.

Anti-CD24 antibodies are well known in the art, and are commercially available. In some embodiments, the antibody is a monoclonal antibody. In some embodiments, the antibody is humanized antibody. In some embodiments, the antibody is an affinity matured antibody. In some embodiments, the antibody is a single-chain antibody. In some embodiments, the antibody is a single-domain antibody.

As used herein, the terms “polynucleotide”, “polynucleotide sequence”, “nucleic acid sequence”, and “nucleic acid molecule” are used interchangeably herein. The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymidine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, circulating nucleic acids, fragments of genomic DNA or RNA, degraded nucleic acids, ribozymes, viral RNA or DNA, nucleic acids of infectious origin, amplification products, modified nucleic acids, plasmidiacl or organellar nucleic acids and artificial nucleic acids such as oligonucleotides.

In some embodiments, the polynucleotide is an RNA interfering (RNAi) polynucleotide. As used herein, the term “RNAi” refers to the process of sequence-specific post transcriptional gene silencing mediated by small interfering RNAs (siRNA). Long double stranded RNA (dsRNA) in cells typically stimulates the activity of a ribonuclease III enzyme referred to as dicer. Dicer is involved in the processing of the long dsRNA into short pieces of siRNA. siRNAs derived from dicer activity are typically about 21-23 nucleotides in length and include duplexes of about 19 base pairs.

The RNAi response also features an endonuclease complex containing a siRNA, commonly referred to as an RNA-induced silencing complex (RISC), which mediates cleavage of single stranded RNA having sequence complementary to the antisense strand of the siRNA duplex. Cleavage of the target RNA takes place in the middle of the region complementary to the antisense strand of the siRNA duplex. Without being bound to any mechanism of processing or action, the present invention relates to double stranded RNAs or antisense RNA molecules, whether processed or not, as a tool for down regulating gene expression.

Gene expression can also be down regulated by microRNAs, single-stranded RNA molecules of about 21-23 nucleotides in length, encoded by genes that are transcribed from DNA but not translated into protein. MicroRNAs base pair with their complementary mRNA molecules and induce mRNA degradation in the RISC complex.

Gene expression can further be down regulated by an antisense oligonucleotide complementary to a region of an mRNA wherein the antisense oligonucleotide is capable of specifically hybridizing with the region of the mRNA, thereby inhibiting the expression of a gene. Thus, the present invention encompasses double stranded RNAs, micro RNAs, antisense oligonucleotides, short hairpin RNAs (shRNAs), each capable of down regulating the expression of CD24.

In some embodiments, the subject is a mammal. In some embodiments, the subject is a human. In some embodiments, the subject has suffered kidney damage. In some embodiments, the subject has suffered kidney injury. In some embodiments, kidney injury comprises kidney necrosis. In some embodiments, kidney injury is acute kidney injury. In some embodiments, the subject suffers from kidney failure. In some embodiments, the subject is at risk for kidney failure. Acute kidney injury is a clinically recognized condition and can be determined using any clinically appropriate measure, including, but not limited to urine test, blood test, glomerular filtration rate test, imaging tests and kidney biopsy.

In some embodiments, prevention occurs after physical damage to a kidney, trauma to a kidney, kidney failure, or another acute kidney injury inducing condition. In some embodiments, after is within 1 hour, 4 hours, 6 hours, 12 hours, 18 hours, 24 hours, 48 hours, 72 hours, 96 hours, 1 week or 2 weeks. In some embodiments, the pharmaceutical composition is administered in this time frame.

According to another aspect, there is provided a method of inhibiting a DAMP in a cell or subject in need thereof, the method comprising administering a CD24 inhibitor.

As used herein, the term “DAMPs” refers to any biomolecule produced by a host organism which can initiate and perpetuate a noninfectious inflammatory response. The terms “damage-associated molecular patterns”, “danger-associated molecular patterns”, “danger signals”, and “alarmin” are used herein interchangeably. Methods for determining the level of DAMPs are common and would be apparent to one of ordinary skill in the art. Non-limiting example for such a method includes, but is not limited to, immunohistochemistry using specific antibodies, such as exemplified herein from CD24.

In some embodiments, a DAMP comprises CD24. In some embodiments, CD24 is a DAMP or an alarmin. In some embodiments, CD24 is not a DAMP. In some embodiments, the DAMP is a renal DAMP.

In some embodiments, the composition comprises or is characterized by a reduced release of a pro-immunogenic cellular component from a damaged cell. In some embodiments, the composition induces a reduction in release of a pro-immunogenic cellular component from a damaged cell. In some embodiments, treatment comprises reduction in release of a pro-immunogenic cellular component from a damaged cell. In some embodiments, release of pro-immunogenic cellular components induces, promotes, facilitates, initiates, propagates, or any combination thereof, an inflammatory response. In some embodiments, treatment comprises reducing inflammation.

In some embodiments, release of pro-immunogenic cellular components induces, promotes, facilitates, initiates, propagates, or any combination thereof, cell death. In some embodiments, cell death comprises necrosis or apoptosis. In some embodiments, treatment comprises reducing cell death.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal.

In some embodiments, the composition is formulated for systemic administration. In some embodiments, the composition is formulated for abdominal administration. In some embodiments, the composition is formulated for subcutaneous administration. In some embodiments, the composition is formulated for intra-peritoneal administration. In some embodiments, the composition is formulated for intravenous administration. In some embodiments, the composition is formulated for administration to a subject.

As used herein, the terms “subject” or “individual” or “animal” or “patient” or “mammal,” refers to any subject, particularly a mammalian subject, for whom therapy is desired, for example, a human.

In some embodiments, the subject is afflicted or at increased risk of developing kidney disease. In some embodiments, the kidney disease is or comprises an acute kidney injury. In some embodiments, the acute kidney injury is or comprises acute tubular necrosis (ATN). In some embodiments, the kidney disease is or comprises a chronic kidney disease.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or formulations prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise”, or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting of”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6^{th} Edition) by Leroy “Skip” G Wade; Organic Chemistry by T. W. Graham Solomons and, Craig Fryhle.

### Materials and Methods

### Animals and Reagents

All animal procedures described in this study were conducted in accordance with the guide for the care and use of laboratory animals published by the Israeli ministry of health and has been approved by the institutional care and use committee. All standard reagents were obtained from Sigma Chemical Co. unless indicated otherwise.

### Animal Model

Study groups comprised 12-14 weeks old wild type (WT) C57BL/6J male mice (Harlan Laboratories, Jerusalem) and CD24 knockout (K/O) mice on a C57/Black background that are bred at the animal facility of the Tel Aviv Medical center, Tel Aviv. These K/O mice are genetically tested on a regular basis by PCR analysis of DNA obtained from tail biopsies at the age 5 weeks, as described previously (Avivi-Arber et al., 2018). Mice were hosted in temperature and humidity-controlled cages with constant light-dark cycles of 12 hours: 12 hours. Mice were provided with food and water ad libitum. Subsequently, animals were segregated into the following groups (10-12 mice per experimental group): Group 1: Control, WT vehicle treated mice; Group 2: CD24 K/O, vehicle treated mice; Group 3: WT folic acid (FA) treated mice, received a single intraperitoneal injection of folic acid of 250 mg/Kg in 0.3 mol/L sodium bicarbonate; and Group 4: CD24 K/O treated mice, as Group 3.

Experimental procedures were performed for each group at days 1, 3, and 7, following the administration of FA. These time points were chosen to capture the insult and the recovery phases of FA-acute kidney injury (AKI).

In a second set of experiments, the inventors studied the effects of anti CD24 antibody treatment in WT mice with AKI (FA-AKI). Mice were injected intraperitoneally with rat anti mouse M1/69IgG2b mAb 10 mg/Kg BW (BioLegend) before induction of AKI, as described previously (Shapira et al., 2011) and were sacrificed on the specified days. A third set of experiments was performed to elucidate the relative contribution of T regulatory cells enhancement for protection against AKI in CD24 K/O mice: An anti-mouse CD25 antibody (PC61) was administered 7 days prior to induction of the renal insult (FA-AKI) to WT mice and K/O mice and the extent of renal injury was compared between the groups. In all experiments, kidneys were perfused in situ with cold saline before removal. Animals are euthanized using CO₂.

### Kidney Function Tests

Plasma samples were collected at the time of sacrifice. Serum creatinine was measured using a standard picrate method (Cayman Chemicals, Michigan, USA) and BUN levels were measured using standard laboratory technique. Immunoperoxidase assay of serum neutrophil gelatinase-associated lipocalin (NGAL), a marker of acute renal tubular injury, was determined by mouse NGAL ELISA kit according to the manufacturer's protocol (Immunology consultants Laboratory, Inc. Portland OR).

### Histological Studies

Following sacrifice, kidneys are bisected, and one half is fixed in 4% formaldehyde (v/v), embedded in paraffin, cross sectioned (4 μm), stained with Periodic Acid Schiff (PAS), and Masson's trichrome to be examined by light microscopy. Tubular injury is evaluated by a pathologist who is blinded to the nature of the samples. Evidence of cell injury (loss of brush border or vacuolization), cell desquamation, and tubular dilation and signs of regeneration is scored on a semi quantitative zero to three scale, and results from each item are added to yield the tubular injury score which has a maximal value of 18.

### Immunohistochemistry

Immunohistochemistry was carried out in paraffin embedded tissue sections (5 μm thick) from the different experimental groups and patients. The samples were incubated with appropriate dilutions of primary antibodies including rabbit polyclonal anti mouse and anti-human CD24, rabbit monoclonal anti-mouse Foxp3 and rabbit monoclonal anti-mouse CD3 to evaluate regulatory T cells renal trafficking, then each section was stained with a panel of antibodies using the ImmPress Reagent (Vector Laboratories) or Optiview DAB IHC detection kit (VENTANA), for mice and human samples, respectively. Control sections consist of staining without primary antibodies and staining with irrelevant primary antibodies (normal rabbit IgG). Scoring of T regulatory cells (Treg) content were determined by overlaying an arbitrary array of gridlines with the aid of image J software and the number of infiltrating Foxp3+ cells/CD3+ cells was determined.

### Western Blotting

Tubular activated caspase-3, Bcl-XL, Myeloperoxidase, IL-33 and TNFα, protein expression were determined by immunoblotting as previously described (Sato et al., 2018). Following sacrifice, renal tubuli from all experimental groups were isolated using a sieving technique as described (Shashar et al., 2019). Tubuli were homogenized in ice-cold PBS lysis buffer (pH 7.4), containing protease inhibitors (1 mM phenylmethylsulphonyl fluoride, 4.5 μM leupeptin, and 5 μM aprotinin) (ICN Biomedicals Inc.), 0.01% Triton X-100 and SDS, then mechanically homogenized and left on ice for 45 minutes. Phosphatase inhibitors: sodium fluoride and sodium orthovanadate (1 mM, Santa Cruz Biotechnology INC. CA, USA) was added to measure phosphorylated proteins. Homogenates were subsequently centrifuged and cell lysates were stored in aliquots in −70° C. A membrane fraction was obtained by adding an equal volume of lysis buffer supplemented by Tween-20 (0.25%). The protein content of each sample was determined by the method of Lowry. Equal amounts of protein (30 μg) was prepared in sample buffer (2% SDS, 0.01% bromophenol blue, 25% glycerol, 62.5 mM Tris HCL, pH 6.8, 5% mercaptoethanol) and analyzed on a 7.5% SDS-PAGE. The gel was transferred onto Hybond ECL nitrocellulose membranes (Amershan Corp.). Following blocking, membranes were incubated with rabbit polyclonal anti-mouse activated caspase-3 antibodies, rabbit monoclonal anti mouse Bcl-XL, (Abcam), mouse monoclonal anti TNF (R&D systems), rabbit polyclonal anti mouse myeloperoxidase and rabbit monoclonal anti-mouse IL-33 antibodies (Abcam), for 1 h at room temperature, washed, and incubated with secondary horse reddish (HRP)-conjugated goat anti-rabbit antibody in PBS-T for 1 h. Membranes were subsequently washed three times, for 5 min each, in PBS-T. Membranes were then stripped and re-probed with monoclonal anti-β actin (MP Biomedicals) or anti Glyceraldehyde-3-Phosphate Dehydrogenase (GAPDH; Santa Cruz Biotechnology) antibodies as an internal control. The immunoreactive proteins were visualized by enhanced chemiluminescence and scanned using MicroChemi Imaging system (DNR Bio-Imaging systems). Band intensity was determined with Image J software. (n=4 different experiments).

### Enzyme-Linked Immunosorbent Assay (ELISA)

Heparinized blood was centrifuged at 3000 rpm for 10 minutes and plasma was harvested. Tubules from each experimental group were homogenized in a lysis buffer with the addition of protease inhibitors (Schwartz et al., 2002). Specific ELISA (R&D systems) was utilized to measure: IL-10; INF-γ; and IL-33, in plasma, and renal tissue, supernatants according to the manufacturer's instructions.

### Example 1

### CD24 and Renal Function in Folic Acid-Acute Kidney Injury (FA-AKI)

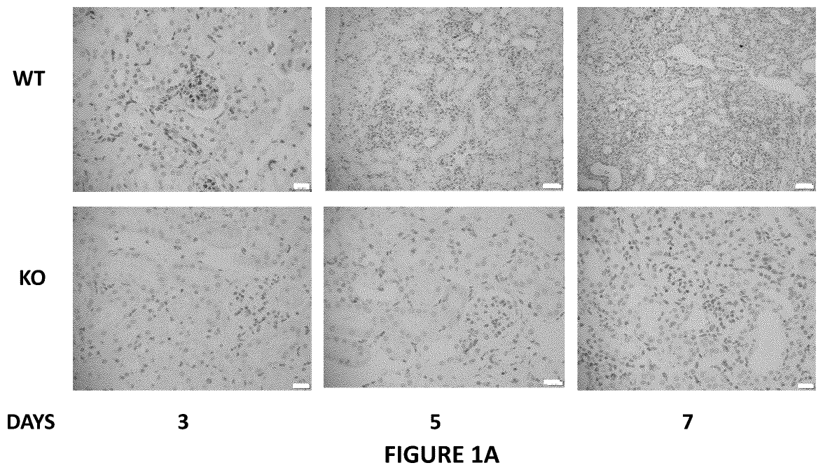

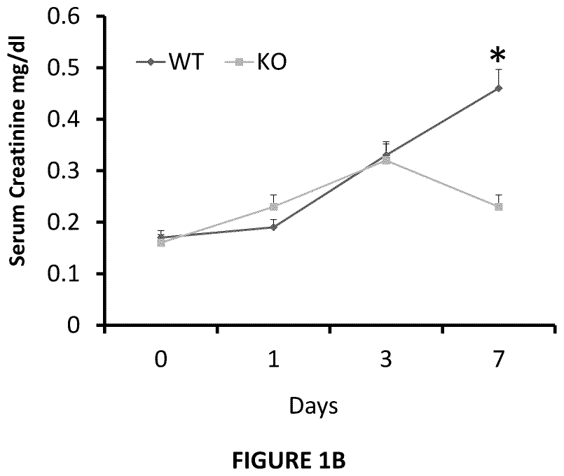

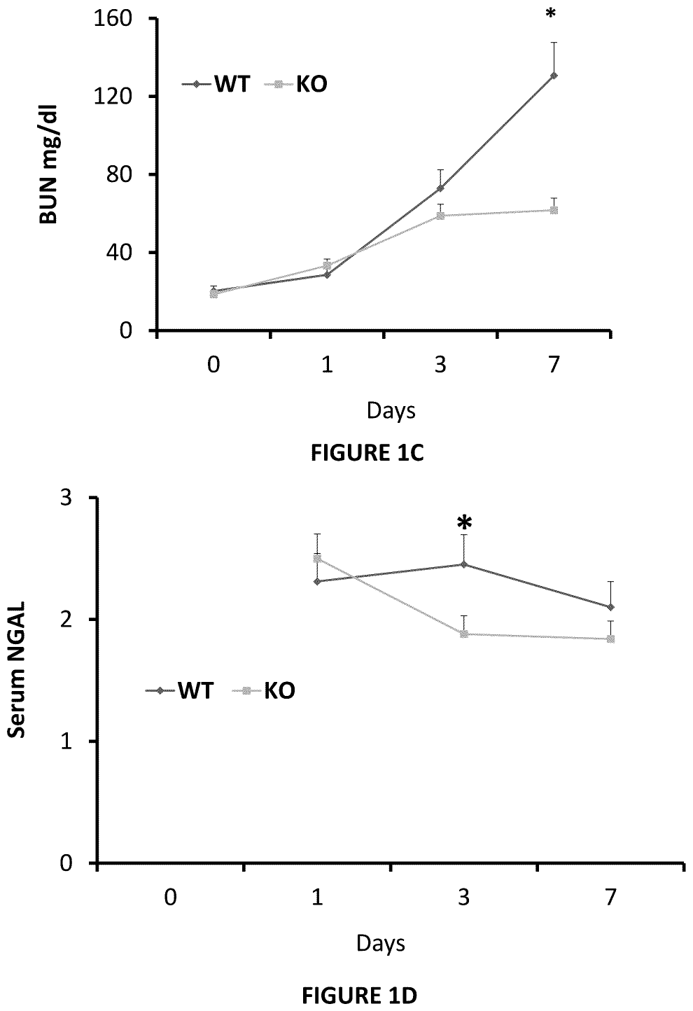

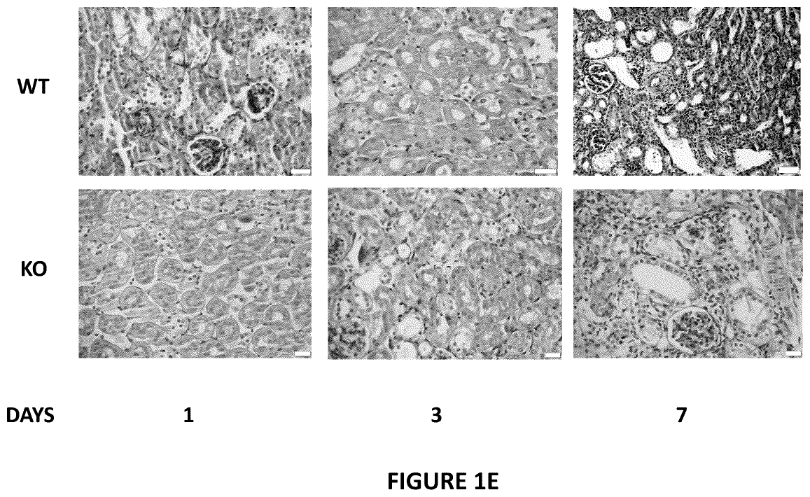

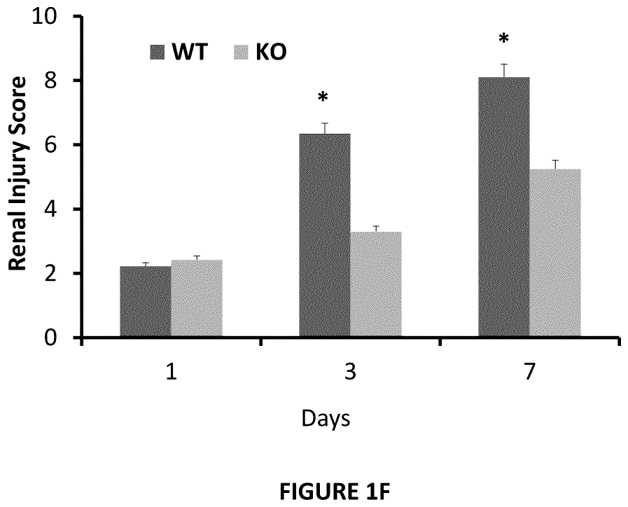

First, the inventors evaluated the presence of CD24 in the different experimental groups. CD24 was absent in WT control mice and in all K/O experimental groups. Progressive positive staining for CD24 restricted to the distal tubular epithelial cells was observed in FA-AKI WT mice. Next, the inventors examined the severity of AKI in response to folic acid administration. The inventors found that on days 1 and 3, serum creatinine and BUN levels increased similarly in wild type and CD 24^{-/-} mice. However, on day 7, renal function continued to deteriorate in WT mice, while it has either remained unchanged (BUN; FIG. 1C) or decreased to baseline values (creatinine; FIG. 1B) in the K/O group. Likewise, FA-AKI WT animals exhibited elevated serum NGAL levels on day 1, which reached a maximum on day 3 and decreased on day 7 (FIG. 1D). Comparably, in the K/O group NGAL increased on day 1 as in the WT group but it started to normalize on day 3 reaching values which were significantly reduced relative to WT group (FIG. 1D). Accordingly, WT and K/O animals manifested similar histologic injury score one day following the folic acid administration (FIGS. 1E-1F). However, it was significantly higher in WT FA-AKI mice on days 3 and 7 compared with the corresponding K/O animals. The most pronounced difference was the patchy peritubular leukocytes infiltration which was significantly reduced in the K/O group (FIGS. 1E-1F).

### Example 2

### CD24 Accelerate Renal Injury

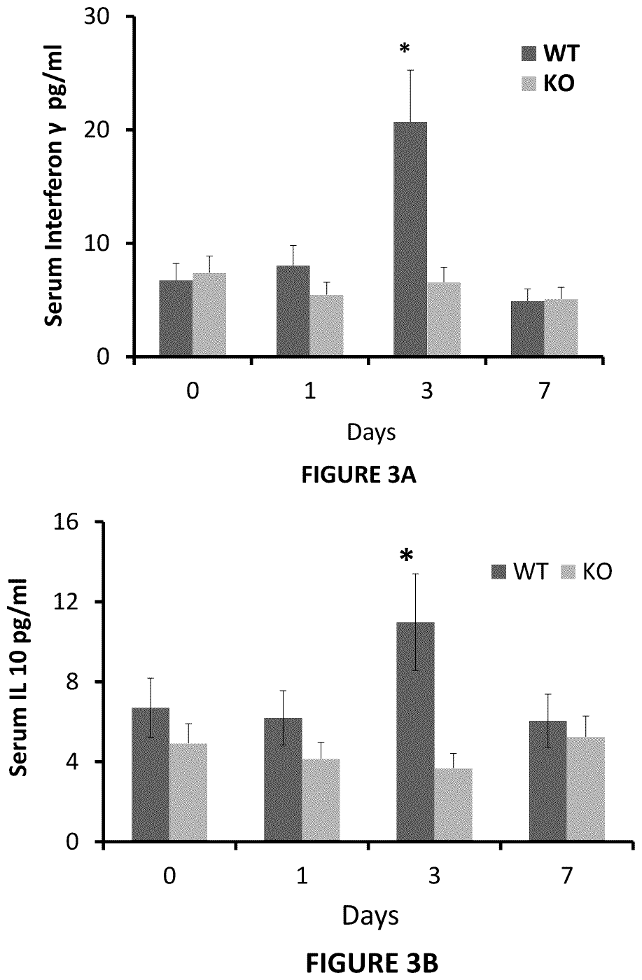

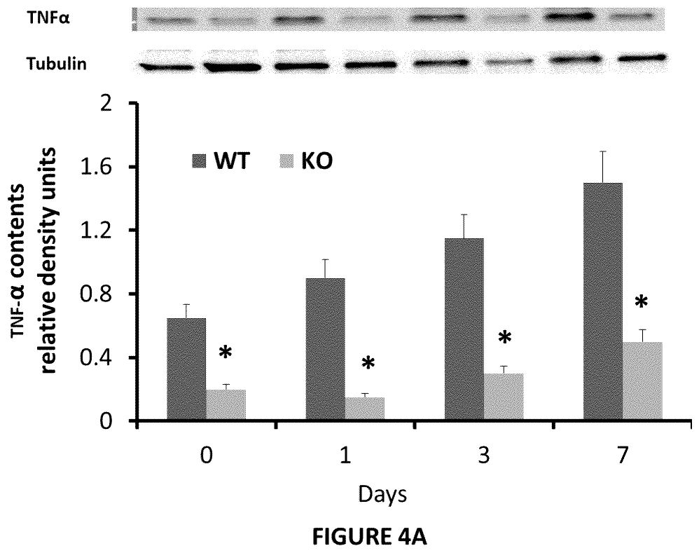

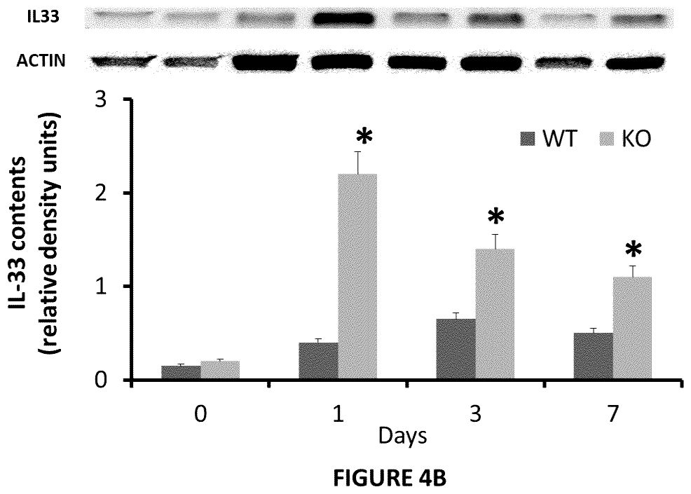

In order to elucidate the mechanism by which CD24 accelerate renal injury in FA-AKI, the inventors have studied the behavior of several cytokines which have been previously shown to play a role in the pathogenesis of acute tubular necrosis (ATN; FIGS. 3-4). The serum levels of INF-γ and IL-10 were significantly higher in the WT animals on day 3 compared with the K/O animals, while on day 7 the differences were not statistically significant. Renal expression of TNFα, evaluated by western blotting, was markedly decreased in the K/O animals on days 1 to 7 following folic acid administration (FIG. 4A).

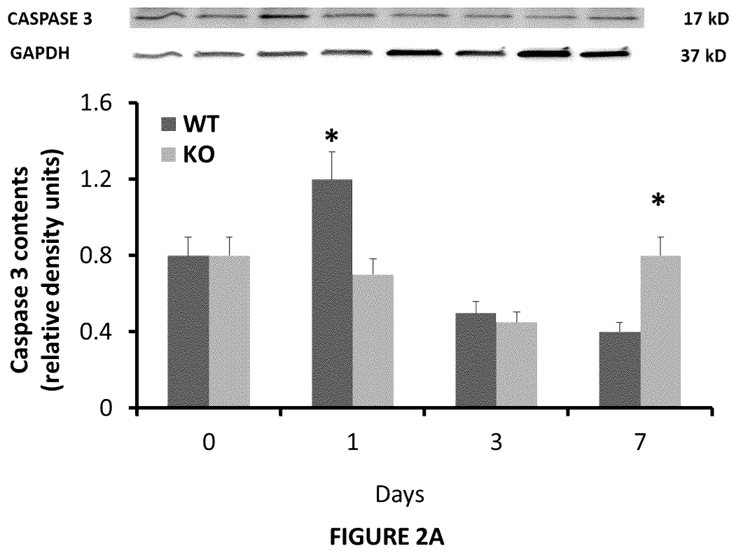

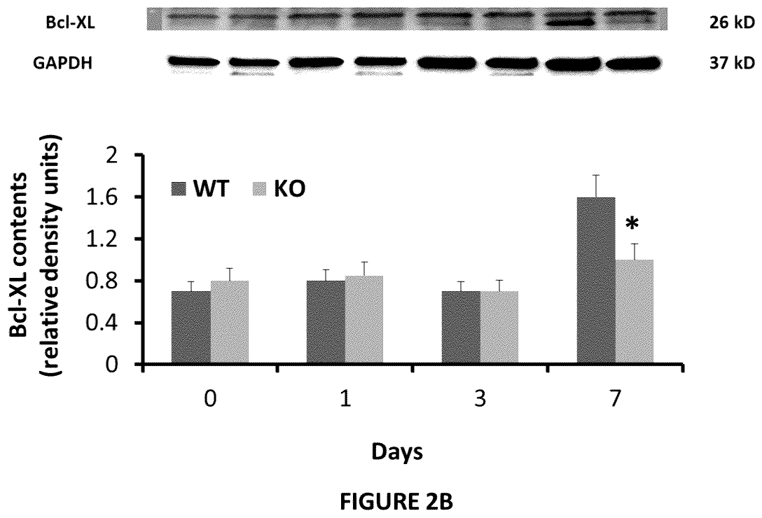

Programed cell death of tubular epithelial cells is a classic hallmark of AKI. To interpret the effects of CD24 on tubular activated caspase 3 (pro-apoptotic) and Bcl-XL (antiapoptotic), the proteins' expression were evaluated by immunoblotting in the different experimental groups (FIG. 2). It was revealed that on day 1 following folic acid activated caspase 3 was significantly over expressed in the WT group, while on day 7 its expression was more pronounced in the K/O animals (FIG. 2A). On the other hand, Bcl-XL exhibited a notable increase in the WT on day 7 compared to the K/O animals (FIG. 2B).

Interleukin 33 (IL-33) is an alarmin, e.g., any endogenous substance which may signal tissue and/or cell damage, which is released by necroptotic cells that can mediate renal injury. In the current experiments the inventors have found that IL-33 protein expression is significantly more elevated at all time points in K/O mice compared with WT mice, reaching a 4-fold difference on day 3 (FIG. 4B). This finding was verified by measuring serum IL-33 in the different experimental groups.

### Example 3

### CD24 Elimination Increases T Regulatory Cells' Infiltration and Splenic Margination

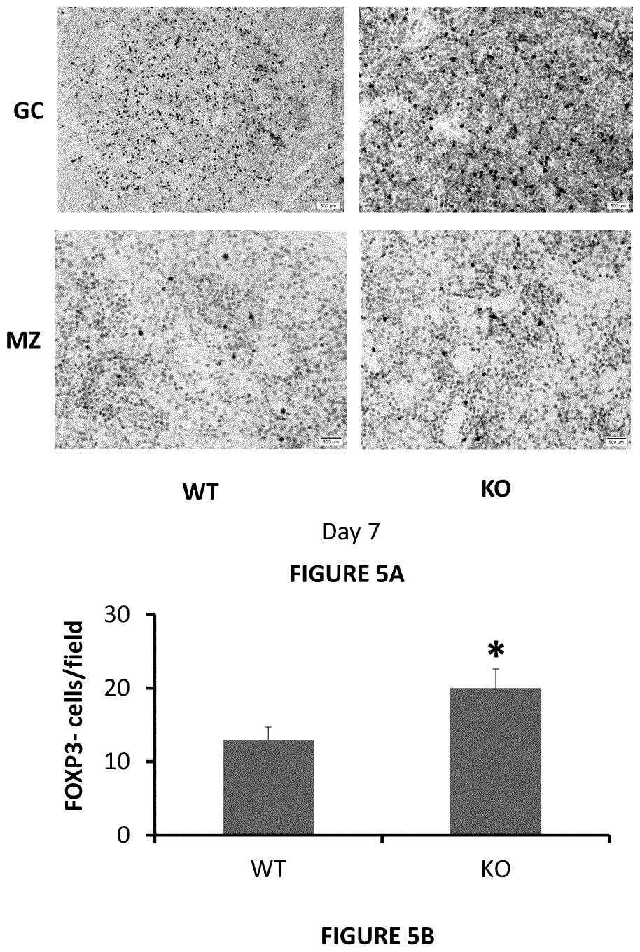

Regulatory T cells (Tregs) are a subtype of T lymphocytes that suppresses innate immunity. Since IL-33 enhances mobilization and renal recruitment of Tregs, the inventors sought to study renal Tregs infiltration and splenic margination on day 7 following FA administration. K/O mice had significantly higher renal Foxp3+/CD3+ ratio than the corresponding WT animals. In the spleen, Foxp3+ positive cells were identified in the germinal centers (GC) in both WT and K/O animals, at a similar extent (FIG. 5A). However, Tregs mobilization evidenced as the number of Foxp3+ positive cells per field outside the GC, was significantly higher in K/O compared to WT mice (FIG. 5B).

### Example 4

### Anti CD24 Therapy Reduces Renal Injury and Improves Renal Function

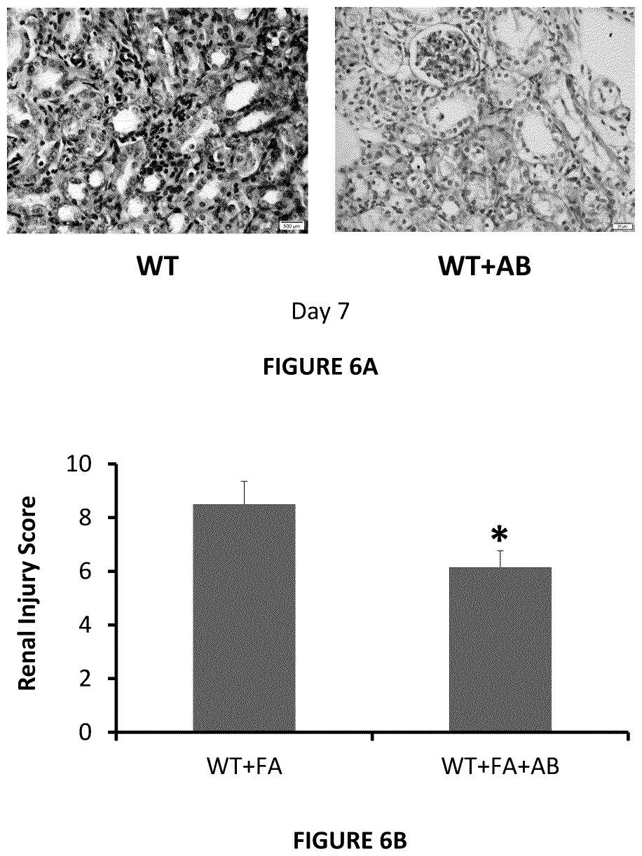

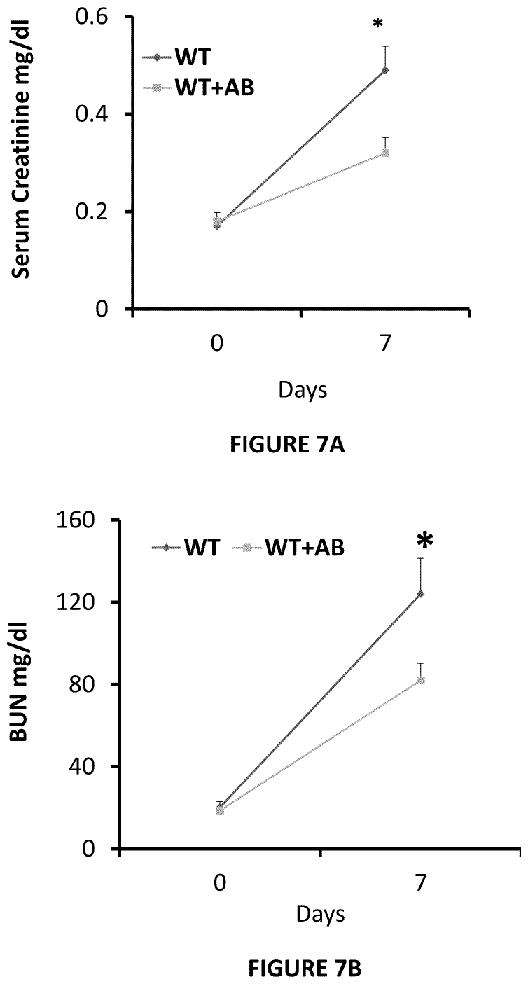

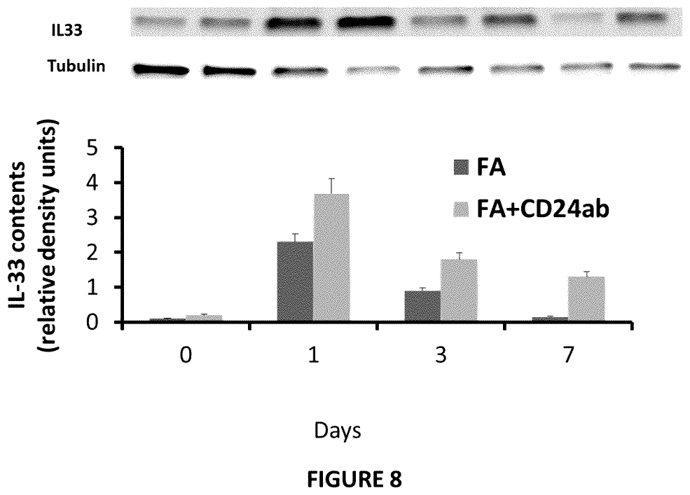

To investigate the effects of anti-CD24 on FA-AKI, anti-CD24 was administered to WT mice before AKI induction and 4 days following the induction (FIGS. 6A-6B). Levels of serum creatinine (FIG. 7A) and BUN (FIG. 7B) on day 7 after FA administration were significantly lower in the anti-CD24 treated group compared to the control group. In addition, CD24 antibodies significantly attenuated leukocytes infiltration. Moreover, IL-33 expression in kidneys from anti-CD24 treated mice was significantly higher compared to the un-treated animals (FIG. 8).

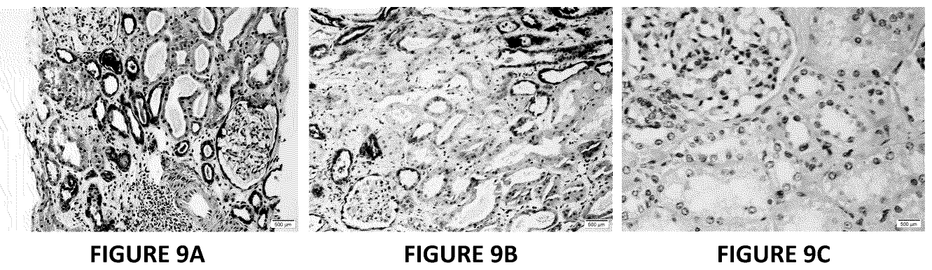

Finally, the inventors examined the presence of CD24 protein in three human kidney biopsies (FIG. 9A-9C). The first patient had no history of renal disease but underwent complete nephrectomy due to renal cell carcinoma (FIG. 9C). The second patient underwent decreased renal transplantation and developed ATN due to prolonged ischemia (FIG. 9B). The last patient presented with severe ANT requiring dialysis support due to prolonged dehydration (FIG. 9A). No staining was observed in the nephrectomized kidney of the first patient while significant expression of CD24 was observed in those two patients with histology confirmed ATN. The positive staining for CD24 was seen solely in the epithelial cells of distal tubules.

In conclusion the current application discloses a new key process, namely CD24 upregulation in the distal nephron, in the pathogenesis of FA-AKI in mice, which adversely affect kidney outcome. CD24 activation is associated with increased renal and systemic inflammation, decreased Tregs recruitment in the kidney, and attenuated apoptotic activity. Therefore, it is suggested that neutralization of CD24, which significantly attenuated the renal insult, may prove to be an effective treatment in AKI.

### Example 5

### The Role of CD 24 Neutralization in Chronic Renal Failure

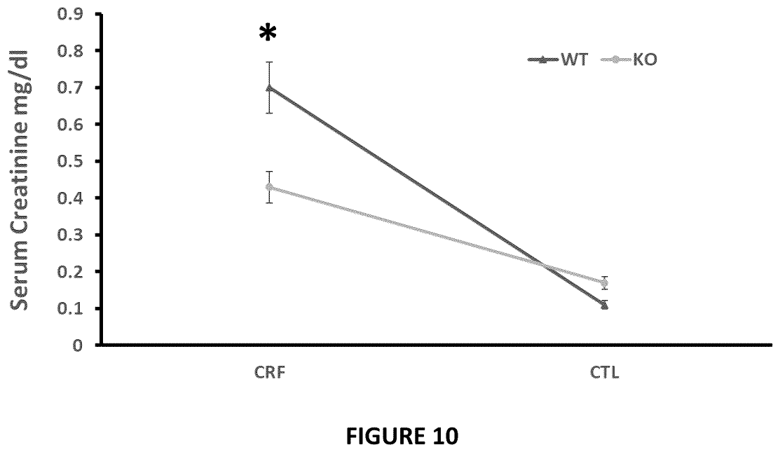

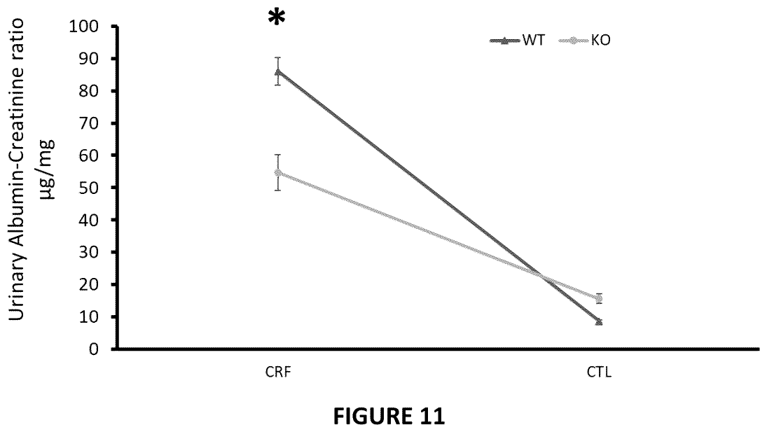

In a different set of experiments CD24 knockout (K/O) and wild type mice (n=8 per group) were subjected to chronic adenine administration as an experimental model of chronic renal failure (CRF). Following one month of treatment, the serum creatinine in the wild type mice increased from a baseline of 0.11 to 0.7 mg/dl compared to a smaller change (0.17 to 0.43 mg %) in the K/O mice (FIG. 10; p<0.01). Urinary albumin excretion increased from 8.7 to 86 μg/mg creatinine in wild type animals while the increase in the K/O was from 15.7 to 54.7 μg/mg creatinine only (FIG. 11; p<0.01). Renal expression of TGF-β and smooth muscle actin (indicators of tissue fibrosis) increased significantly more in the wild type mice compared to the K/O animals with CRF (data not shown). In addition, the expression of FGF23 (a marker of the metabolic bone disease of CRF) in renal tissue from wild type mice was significantly increased compared to K/O animals with CRF (data not shown).

The inventors conclude that the absence of CD24 in mice with chronic renal failure significantly attenuates the severity of the renal disease in mice, hence treatment with a CD24 inhibitor, as disclosed herein is therapeutically relevant in the context of chronic renal disease.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

What is claimed is:

1. A method for treating or preventing a kidney disease or injury in a subject in need thereof, the method comprising administering to said subject a therapeutically effective amount of pharmaceutical composition comprising a CD24 inhibitor, thereby, treating or preventing a kidney disease or injury in the subject.

2. The method of claim 1, wherein said kidney disease or injury comprises any one of: acute kidney injury (AKI), and chronic kidney disease.

3. The method of claim 1, wherein said treating or preventing comprises reducing a level of at least one cytokine selected from the group consisting of: interleukin-10 (IL-10), interferon gamma (INF-γ), tumor necrosis factor alpha (TNFα), and any combination thereof, in said subject.

4. The method of claim 1, wherein said treating or preventing comprises increasing a level of IL-33, in said subject.

5. The method of claim 1, wherein said treating or preventing comprises reducing levels of serum creatinine, blood urea nitrogen (BUN), urinary albumin to creatinine ratio, or any combination thereof, in said subject.

6. The method of claim 1, wherein said treating or preventing comprises increasing splenic margination of T regulatory cells (Tregs), in said subject.

7. The method of claim 1, wherein said treating or preventing comprises increasing renal infiltration of Tregs, in said subject.

8. The method of claim 7, wherein said Tregs are Foxp3+ Tregs.

9. The method of claim 2, wherein said AKI comprises acute tubular necrosis (ATN).

10. The method of claim 1, wherein said pharmaceutical composition further comprises a pharmaceutically acceptable carrier.

11. The method of claim 1, wherein said CD24 inhibitor is a polypeptide or a polynucleotide.

12. The method of claim 11, wherein said polypeptide is an antibody.

13. The method of claim 12, wherein said antibody is a blocking or inhibitory antibody.

14. The method of claim 12, wherein said antibody is not cytotoxic.

15. The method of claim 11, wherein said polynucleotide is an RNA interfering polynucleotide.

16. The method of claim 1, wherein said treating or preventing comprises reducing release of pro-immunogenic cellular components from damaged cells, in said subject.

17. A method for inhibiting a damage associated molecular pattern (DAMP) in a subject in need thereof, the method comprising administering to said subject a therapeutically effective amount of a pharmaceutical composition comprising a CD24 inhibitor and a pharmaceutically acceptable carrier, thereby inhibiting a DAMP in the subject.
